(19) United States
(12) Patent Application Publication
(10) Pub. No.: US 2025/0354967 A1
(43) Pub. Date: Nov. 20, 2025

(54) METHOD FOR CALIBRATING SENSOR UNITS FOR DETERMINING A CONCENTRATION OF A GAS IN A GAS MIXTURE

(71) Applicant: BAUER KOMPRESSOREN GmbH, Münich (DE)

(72) Inventors: Robert KAMPFL, München (DE); Thomas BURMEISTER, Sachsenkam (DE); Johannes HUBER, Friedberg (DE)

(73) Assignee: BAUER KOMPRESSOREN GmbH, Münich (DE)

(21) Appl. No.: 18/874,534

(22) PCT Filed: Jun. 14, 2023
(86) PCT No.: PCT/EP2023/065908

§ 371 Date: Dec. 12, 2024

(30) Foreign Application Priority Data
Jun. 15, 2022 (DE) 10 2022 115 062.3

(51) Int. Cl. G01N 33/00

(52) U.S. Cl. CPC G01N 33/0006; G01N 33/004; G01N 33/0073

(57) ABSTRACT

The present invention relates to a method for calibrating at least one sensor unit which is provided for determining a concentration of a gas in a gas mixture, wherein a limit value concentration (X_{G}) is predetermined, comprising an initial calibration of the sensor unit at at least two concentrations (X₁, X₂) of the gas in a respective calibration gas mixture, at least comprising the steps of determining an initial zero point output (Y₁) of the sensor unit with a first calibration gas mixture, determining a further output (Y₂) of the sensor unit with a second calibration gas mixture, and forming an output straight line of the sensor unit based on the initial zero point output (Y₁) and the further output (Y₂), wherein the output line has an initial straight line slope (α). According to the invention, the method further comprises a recalibration at a later point in time, comprising the steps of determining a limit value output (Y_{G}′) of the sensor unit with a limit value gas mixture in which the concentration (X_{G}) of the gas substantially corresponds to the limit value concentration (X_{G}), and forming an updated output straight line of the sensor unit based on the limit value output (Y_{G}′) and the initial zero point output (Y₁) or based on the limit value output (Y_{G}′) and the initial line slope (α).

### DESCRIPTION

The present invention relates to a method for calibrating at least one sensor unit that is provided for determining a concentration of a predetermined gas in a gas mixture, as well as to an analysis unit for gas mixtures, the control unit of which is designed to carry out such a method.

It is known that certain gas mixtures, for example when they are filled into compressed gas reservoirs, such as breathing air in breathing air cylinders, or when they are withdrawn from such reservoirs before further use, must be constantly tested for their quality and composition, and in particular for the presence of impurities. Such impurities must not exceed specified limit values with regard to their respective concentrations in the corresponding gas mixture, and it must be ensured that if a limit value is exceeded, then suitable countermeasures are adopted immediately, for example stopping operations.

For this purpose sensor units are used, which in the example mentioned above are interposed between a compressor for compressing the gas mixture to be filled and the compressed gas reservoir, and which are each provided and designed to determine a concentration of a predetermined gas in the gas mixture. In this case, in the example just described several sensor units can be provided, each of which can monitor a component of the gas mixture in terms of its concentration. This can relate to both desired and undesired components of the gas mixture, so that the quality and composition of the gas mixture as a whole can be permanently monitored both in terms of its desired components and undesired impurities.

It is known that when using such sensor units, a readjustment of the sensor outputs or the raw values provided by the sensor units may be necessary after a certain length of time in order to compensate for normal changes in the measuring section and the sensors, and to permanently ensure the accuracy of the concentrations determined by means of the sensor units. Although, as has just mentioned, this strictly speaking involves an adjustment of the corresponding sensor units, the term “calibration” has also become established in the technical language for such methods, and this term will be used throughout the following to describe the method according to the invention.

For this purpose, the prior art involves comparing the raw values provided by the sensor units at two or more defined points with a gas mixture in which the corresponding gas to be detected by the sensor unit is present as a component in a known concentration, which is usually referred to as “two-point calibration”. However, the running costs for calibrating or adjusting such sensor units are very high, since corresponding calibration gas mixtures with a defined concentration of the gas to be detected are very expensive and the process must be carried out or at least monitored by trained service personnel. There is therefore a need for a process and an analysis unit with which such a calibration can be carried out more easily and with cost savings, while ensuring however that the corresponding sensor unit still provides sufficiently accurate data after the calibration.

In order to achieve this object, the invention proposes a method for calibrating at least one sensor unit which is provided for determining a concentration of a predetermined gas in a gas mixture, wherein a maximum limit concentration is specified which must not be exceeded by the predetermined gas in the gas mixture, said method comprising an initial calibration of the sensor unit at at least two predetermined concentrations of the predetermined gas in a respective calibration gas mixture, at least comprising the steps of determining an initial zero point output of the sensor unit with a first calibration gas mixture in which the concentration of the predetermined gas is substantially zero, determining a further output of the sensor unit with a second calibration gas mixture in which the concentration of the predetermined gas is greater or less than the maximum limit concentration, and forming an output straight line of the sensor unit based on the initial zero point output and the further output, wherein the output line has an initial straight line slope. Furthermore, the method according to the invention comprises a recalibration at a later point in time, comprising the steps of determining a limit value output of the sensor unit with a limit value gas mixture in which the concentration of the predetermined gas substantially corresponds to the limit value concentration, and forming an updated output straight line of the sensor unit based on the limit value output and the initial zero point output or based on the limit value output and the initial straight line slope.

Accordingly, according to the invention a two-point calibration is carried out only during the initial calibration of the at least one sensor unit, in which on the one hand a zero point output of the sensor unit and on the other hand a further output of the sensor unit at a higher concentration of the gas to be determined in the gas mixture is measured, wherein the original output line of the sensor unit is then formed based on these two measuring points. In this connection, the second calibration gas mixture can preferably be selected such that the concentration of the predetermined gas is above the limit value in order to have available one data point below and one above the limit value and to be able to establish a suitable output straight line. At this point it should already be noted that the output behavior of such sensor units does not necessarily have to be strictly linear, but can usually be approximated sufficiently well by a straight line. If necessary, additional safety factors can then be introduced in the regular operation of the sensor unit in order to correct the measured concentration values, so that exceeding the required limit values can also be ruled out again at this point with even greater certainty.

Furthermore, it should be noted that the determination of the zero point output can also take place with a first calibration gas mixture which contains the predetermined gas in a low concentration, for example if providing a corresponding gas mixture without the corresponding gas would be economically or technically too expensive or complicated or even impossible. Accordingly, the expression “substantially zero” in connection with a concentration of a gas in a gas mixture is understood within the scope of the present invention to mean that the smallest possible value should be aimed for that is technically and economically feasible and that allows a sufficient differentiation from the concentration of the corresponding gas in the second calibration gas mixture.

In the subsequent recalibration of the at least one sensor unit, according to the invention a so-called “limit value single-point calibration” is employed however, in which the output of the sensor unit is determined essentially at that point in the concentration spectrum of the corresponding gas at which the limit value of this gas is located in the gas mixture to be checked during a regular operation of the sensor unit.

Based on this individual determination of the limit value output when recalibrating the sensor unit, it can therefore be ensured that the sensor output is known with the highest possible accuracy specifically at this limit value of the concentration of the corresponding gas in the gas mixture, whereas correspondingly greater inaccuracies can be accepted for example at an upper end of the possible measuring range of the corresponding sensor unit, since it is assumed that at this point there is a large distance to the essential limit value and consequently no high degree of accuracy is required at this point in the concentration spectrum. Ultimately, the present invention is based on the knowledge that in such a scenario, a sensor unit essentially only has to determine whether a predetermined limit value of a gaseous component of a gas mixture has or has not been exceeded, and therefore whether the gas mixture is or is not suitable for its intended purpose.

Accordingly, according to the invention it is possible to form the updated output line based on the limit value output determined during the recalibration and the initial zero point output of the sensor unit, which will lead to a changed straight line slope of the output line if the sensor output changes over time, while on the other hand it is also conceivable to use the originally determined initial straight line slope and the limit value output recorded during the recalibration to form the updated output line, which will consequently, inter alia, shift the zero point output on the output line.

The outputs of such sensor units are typically electrical voltages or currents, which are expected to exhibit an at least approximately linearity of their outputs as regards the concentration of the gas to be determined in their envisaged measuring ranges. It should furthermore be noted that the recalibration within the scope of the present invention can take place after a predetermined time or within the framework of a predetermined time period or also repeatedly during a respective renewed operation of the corresponding sensor unit.

In any case, considerable savings can be achieved by the method according to the invention, since only a single limit gas mixture is required for the recalibration, compared to at least two calibration gas mixtures required in two-point calibration methods known from the prior art.

Since significant changes in the sensitivity or sensor outputs of the sensor units considered here can occur over their service life depending on the concentrations to be determined, the method according to the invention can, after forming the updated output line, further include a step of checking whether the straight line slope of the updated output line and/or a zero point value of the updated output line lie within a predetermined range of values. In this way cases can for example be intercepted in which the sensor output has decreased to such an extent that when an updated output line is formed, the initial zero point output is greater than the newly determined limit value output, which would lead to a negative line slope, which in turn would lead to incorrect results when monitoring the limit value of the corresponding gas in the gas mixture during regular operation of the sensor unit. By such a plausibility check of the updated output line with regard to its slope and/or its zero point value, such problems can therefore be excluded from the outset, and if one of the values mentioned is not within the predetermined value range, corresponding countermeasures can then be adopted, for example issuing a warning or closing down the corresponding at least one sensor unit.

Furthermore, according to the invention the predetermined gas can be present in the second calibration gas mixture at a concentration which corresponds to an upper limit of the envisaged measuring range of the corresponding sensor unit. In this way, during the initial calibration of the sensor unit the zero point output and a further output of the sensor unit as remote as possible from this zero point are evaluated, which accordingly leads to an initial output straight line that is as precise as possible.

Furthermore, the initial calibration can include a determination of multiple further outputs with different gas mixtures, in which the concentration of the predetermined gas is different and is greater than zero, as well as the formation of a compensation output line of the sensor unit based on the initial zero point output and the multiple further outputs. This procedure can also be used to determine the most precise possible initial output line of the sensor unit, whereby statistical and systematic fluctuations are reduced by recording multiple further outputs with different gas mixtures. Different methods can then be used to form the corresponding compensation output line, such as for example linearization using the least squares method or an adapted variant thereof, in which for example the zero point can be fixed or given a greater weighting.

Furthermore, in the method according to the invention a plurality of sensor units can be calibrated simultaneously during the initial calibration and/or recalibration, in which the respective gas mixtures contain, in suitable concentrations, a corresponding plurality of gases to be determined. In practice this means that a smaller number of gas mixtures to be used for the calibration need to be provided, for example by providing a first calibration gas mixture in which a plurality of predetermined gases to be detected by individual sensor units are each present at zero concentration, and a second calibration gas mixture in which the corresponding gases are each present at a respective higher concentration, which in each case enables an initial output line to be formed for each sensor unit and each of the gases to be detected in the gas mixture.

This can result in significant savings in costs and effort in systems in which a plurality of corresponding sensor units are integrated, and the corresponding sensor outputs need only be temporarily stored in a suitable manner and then processed. In this connection, it is only necessary to ensure that the sensor units to be calibrated do not have any cross-sensitivities for several of the components contained in the gas mixture, though this naturally depends however on the types of predetermined gases and the respective design of the individual sensor units. However, since such cross-sensitivities are known for sensor units used in such applications, an optimal combination of gaseous components in the individual calibration gas mixtures or limit gas mixtures can be ensured in each case for corresponding calibration processes, in which many possible combinations are conceivable, for example a completely purified gas in which, as already mentioned, none of the components to be detected are present at all, and/or a plurality of limit value mixtures with different components, provided that these can be produced in a suitable manner and used with the types of sensor units that are employed.

In practice, the method according to the invention can be used in particular when the gas mixture is breathing air as already mentioned above, and/or the predetermined gas is CO, CO₂ or O₂. For breathing air to be filled into breathing air cylinders, the DIN EN 12001 standard in particular is applicable, which specifies corresponding limit values for the aforementioned gaseous components, for example 500 ppm for CO₂ and 5 ppm for CO. These specified values therefore correspond to the limit values already discussed several times above, in which the recalibration of the corresponding sensor units would have to take place within the scope of the limit value single-point calibration in this example.

Furthermore, after the initial calibration and/or recalibration the method according to the invention can include a check of the calibration with a test gas mixture in which the concentration of the predetermined gas is slightly above the limit value concentration. In this way it can be ensured that even a small overshoot of the corresponding limit value is also reliably detected, and accordingly the corresponding sensor unit fulfills its task in the intended manner. In a specific example, the concentration of the predetermined gas in the test gas mixture can be about 10-30% above the corresponding limit value concentration, i.e. in the case of CO in breathing air is 6 ppm compared to the limit value of 5 ppm.

Furthermore, the method according to the invention can include monitoring a flow rate of the respective gas mixture through the at least one sensor unit. This further aspect of the invention relies on the fact that gas canisters are generally used for the calibration of such sensor units, since these are easy to transport and contain enough gas for a corresponding calibration. The commercially available pressure reducers for such gas canisters used in this connection have the disadvantage however that the flow is determined by the canister pressure, and therefore it can happen during calibration that the flow does not correspond to the later operating conditions of the sensor unit to be calibrated in a productive operation. Consequently, if there is a deviation from the intended flow the calibration could be inaccurate and the corresponding sensor unit could fail in a possible subsequent test.

Specifically, in order to carry out this process step the flow through the corresponding sensor unit could be monitored via a measured pressure difference between an employed hose system and the ambient air, so that at any time during the calibration it is known which flow is currently in the sensor.

If the flow rate falls below and/or exceeds a respective limit value flow rate for a predetermined time interval, a warning could be issued or the calibration could be automatically stopped. The purpose of having this predetermined time interval is to ensure that the calibration is not stopped every time the defined tolerance range is briefly exceeded or undershot, which would make the process significantly less efficient. Accordingly, values of around 10 seconds could be envisaged for example, after which the calibration would be stopped if the flow rate exceeds or falls below the limit value.

Furthermore, the present invention relates to an analysis unit for gas mixtures, comprising at least one sensor unit which is designed to output sensor data which represent a concentration of a predetermined gas in a gas mixture, and a control unit which is operatively coupled to the at least one sensor unit and is designed to determine in a working mode the concentration of the predetermined gas based on the sensor data, wherein the control unit is further designed to be switchable between said working mode and a calibration mode and to carry out in said calibration mode the steps of the method according to the invention described above. In this case different degrees of automation of the control unit or of the method for calibrating the at least one sensor unit are conceivable, for example it can be envisaged that a human operator can actuate the individual method steps individually or also that the method can be carried out largely automatically, in which case a human operator simply has to perform a monitoring function.

Furthermore, the control unit of the analysis unit can be designed to issue a control command to open a flush valve or shut down a compressor in the working mode if it detects that a limit value concentration of the predetermined gas of the at least one sensor unit has been exceeded. This thereby ensures that if for example in the afore-mentioned example of filling breathing air one of the limit values to be observed is exceeded, the filling of the corresponding breathing air cylinder is immediately stopped and a filling of contaminated breathing air is thereby reliably avoided.

Furthermore, the analysis unit can be provided with a gas reservoir which contains a test gas mixture in which the at least one predetermined gas is present in an amount slightly above its limit value concentration, and the control unit can be designed to automatically switch to a test mode after a predetermined time interval or when the analysis unit is started up again, and to carry out a test of the at least one sensor unit with the aid of the test gas mixture contained in the gas reservoir. In this case the start-up of the analysis unit can for example be associated with the start-up of a compressor, the output gas from which is monitored by the analysis unit.

This automated solution, which automatically charges the sensor system with the test gas mixture after a predeterminable interval and thus tests the functionality of at least one sensor unit, can save maintenance costs and avoid the use of service technicians. The corresponding control is performed directly via the electronic control unit of the system, and after the test interval set in the corresponding software has expired the connection to a current pressure source, such as the compressor already mentioned several times above or even a breathing air cylinder to be checked, can be disconnected and a corresponding solenoid-operated valve for the gas reservoir, for example a test gas bottle, can be opened. The test gas mixture then flows at a preset flow rate through the at least one sensor unit to be tested and the control unit can monitor whether the sensor values that are output are within a predetermined tolerance range. If this is not the case, the compressor can be switched off or a corresponding warning can be issued. In order to prevent untested gas mixtures being filled during this automatic sensor test, the compressor can be switched off or an intermediate flush valve can be opened, depending on the operating mode of the system.

Since during the initial or renewed calibration and/or testing, the gas mixture compressed for a filling procedure, for example by a compressor monitored by the at least one sensor unit, cannot be monitored by the sensor unit, measures can then be adopted according to the invention to prevent the filling of gas mixtures that are not monitored in this way. In particular, the corresponding compressor can be switched off during the initial or renewed calibration and/or testing, a flush valve between the compressor and a corresponding compressed gas reservoir can be opened, and/or a display can be adjusted for an operator so that the latter is informed about the instantaneous execution of the corresponding process step and can initiate corresponding measures manually if necessary.

Accordingly, the test mode just described corresponds in principle to the working mode of the analysis unit, although if a specified limit value is exceeded this does not mean that the system has to be shut down, but rather that the function of the system can be verified.

As already mentioned above, the or at least one of the sensor units may be an electrochemical sensor unit, wherein such sensor units function in such a way that they contain a chemical substance that reacts with the predetermined gas to be analyzed and thereby generates an electrical signal.

The present invention furthermore relates to a compressor for compressing breathing air for filling into a compressed gas reservoir, comprising an analysis unit of the type just described, which is designed to monitor the emitted compressed breathing air for a concentration of at least one predetermined gas during an operation of the compressor. For this purpose the control unit of the analysis unit can be operatively coupled to a control unit of the compressor or integrated therewith in order to achieve an enhanced integration of the system and, if necessary, to be able to shut down the compressor immediately in an efficient manner if for example a limit value is exceeded.

Further features and advantages of the present invention will become more apparent from the following description of embodiments thereof when considered together with the accompanying figures. These show in detail:

FIG. 1 is a schematic block diagram of a device according to the invention;

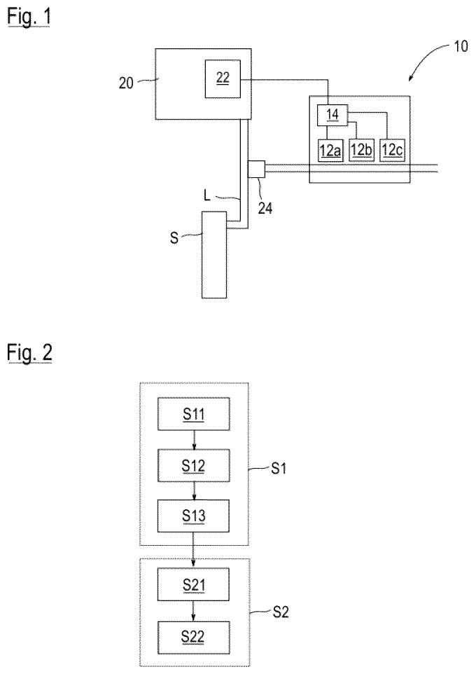

FIG. 2 is a flow chart of a method according to the invention; and

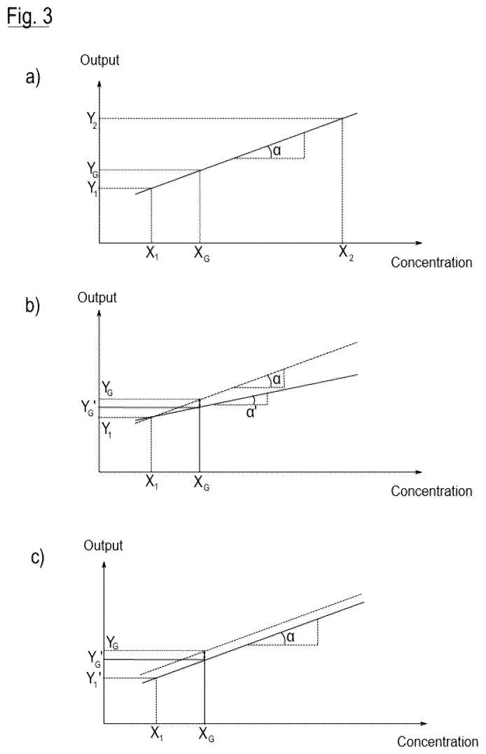

FIG. 3 shows several diagrams to illustrate the process of FIG. 2.

In FIG. 1 an analysis unit for gas mixtures according to the invention is first of all shown schematically and generally designated with the reference number 10. The analysis unit 10 comprises a plurality of sensor units 12a to 12c, which are provided and designed for example for a respective measurement of a concentration of CO, CO₂ and O₂ in breathing air intended for filling into breathing air cylinders. The analysis unit 10 furthermore comprises a control unit 14, which is operationally coupled to each of the sensor units 12a to 12c and can process the corresponding sensor outputs in a working mode in order to carry out a monitoring of predetermined limit values of the corresponding components of the breathing air to be filled.

Furthermore, FIG. 1 shows a compressor 20, which supplies the already mentioned compressed breathing air to be filled and also comprises a control unit 22, which in the illustrated embodiment is operationally coupled to the control unit 14 of the analysis unit 10. The breathing air compressed by the compressor 20 is filled during regular operation of the compressor into a breathing air cylinder S acting as a compressed gas reservoir by means of a line system L, shown only schematically, wherein a part of the compressed breathing air is removed via a pressure reducer 24 and is passed to the analysis unit 10 for analysis by means of the sensor units 12a to 12c. In an alternative variant of the device shown, the pressure reducer 24 could also be dispensed with so as to subject the sensor units 12a to 12c directly to high pressure. It is further understood that, in particular, the line system L can include further components not shown here, for example a gas drying unit or safety valves, which however are not important in the context of the present invention.

If a predetermined limit value of a concentration of one of the above-mentioned components of the breathing air to be filled is exceeded and is accordingly detected by the sensor units 12a to 12c, a warning can be issued by the control unit 14 and/or an instruction to stop the operation of the compressor 20 can be transmitted immediately to its control unit 22. Alternatively, a flush valve, not shown, could also be opened so that the compressed breathing air is also not filled into the breathing air cylinder S, though the operation of the compressor 20 does not have to be stopped. Furthermore, in certain embodiments of the present invention the analysis unit 10 can also be integrated directly into the compressor 20, so that the two control units 14 and 22 could also be formed as one unit.

In order to calibrate the sensor units 12a to 12c according to the invention, a method according to FIG. 2 can now be carried out, which comprises an initial calibration S1 and a recalibration S2, wherein the initial calibration S1 can for example take place at the factory before delivery of the corresponding analysis unit 10, while the recalibration S2 can be carried out in situ on analysis units that have already been installed and are operating. The corresponding method can be carried out separately for each of the sensor units 12a to 12c, or several of the sensor units 12a to 12c can be calibrated simultaneously.

In this case the initial calibration S1 first of all involves determining an initial zero point output of the corresponding sensor unit with a first calibration gas mixture, in which the concentration of the predetermined gas is substantially zero, in a step S11. This step can be regarded as carrying out the determination of the initial zero point output of all of the sensor units 12a to 12c simultaneously, provided that a corresponding calibration gas mixture is available, in which the concentration of all of the gases to be determined by the sensor units 12a to 12c is zero.

Then, in step S12, a further output of the corresponding at least one sensor unit is determined with a second calibration gas mixture, in which the concentration of the predetermined gas is smaller than or preferably larger than the limit value concentration. In this case also a parallel determination of further outputs of a plurality of the sensor units 12a to 12c can take place, provided that corresponding calibration gas mixtures are available that contain several of the individual gases in suitable concentrations, and if the sensor units 12a to 12c that are used do not show any cross-sensitivities. It should be noted here that step S12 can also be carried out several times with different gas mixtures in which the concentration of the predetermined gas is different in each case but is greater than zero, following which a plurality of corresponding values can then be processed subsequently.

In step S13 an output straight line of the corresponding sensor unit is then formed based on the zero point output determined in steps S11 and S12 and the at least one further output, wherein the output line has an initial line slope α. This initial output line is used after a first operation of the corresponding sensor unit, in which connection it is to be expected that the properties of the sensor units 12a to 12c will change over time as regards their sensor outputs, and consequently a recalibration will be necessary at regular intervals.

For this purpose, during the recalibration S2 a limit value output of the corresponding sensor unit can be determined with a limit value gas mixture in step S21, in which the concentration of the predetermined gas substantially corresponds to a limit value concentration in the manner described above by way of example involving breathing air. Also, at this point the limit value gas mixture can again contain several of the gases to be detected in their respective limit value concentrations, so that a determination of limit value outputs from a plurality of the sensor units 12a to 12c can again be carried out simultaneously.

Next, in step S22 an updated output straight line of the corresponding sensor unit is determined based on the newly determined limit value output and the initial zero point output or based on the limit value output and the initial line slope. In this way it can be can ensured that in the vicinity of the corresponding limit value concentration of the gas to be detected by means of the respective sensor unit in a gas mixture to be filled, a high degree of accuracy is achieved in this respect regardless of whether the limit value is exceeded or undershot.

The method carried out in the arrangement of FIG. 1 in the manner illustrated in FIG. 2 will now be explained by way of example with the aid of the diagrams in FIG. 3. Here, the diagram shows a) a sensor output on the y-axis, which is plotted against a concentration of a corresponding gas in a gas mixture to be examined on the x-axis. Depending on the type of sensor used, the raw sensor value can be, for example, a voltage in millivolts (mV) or a current in milliamperes (mA). The two values X₁ and X₂ marked on the x-axis correspond to a concentration of 0 or a concentration that represents an upper limit of the measuring range of the corresponding sensor unit. In addition, a limit value concentration is marked by the x-value X_{G}. At the points X₁ and X₂, a zero point output and a further output of the sensor unit can now each be determined during the initial calibration of the corresponding sensor unit, which accordingly correspond to the output values Y₁ and Y₂. By drawing a straight line having the already-mentioned slope α through the points (X₁, Y₁) and (X₂, Y₂), the limit output YG associated with the limit value concentration X_{G} can be derived.

It is to be expected that, with commonly used sensor units, the outputs shown on the y-axis for given concentrations on the x-axis will change, and in particular will decrease over time. A recalibration is therefore necessary after a certain time span or according to a certain period of time, as described above.

Since the sensor units 12a to 12c in the application discussed here primarily have to monitor whether the limit value concentration X_{G} is undershot or exceeded, according to the invention such a recalibration can now take place in a simple and efficient manner by means of a limit value one-point calibration at the concentration value X_{G}, in which the limit value output Y_{G}′ is determined in the aforementioned manner. The two possible ways of deriving a corresponding updated output straight line can be understood with the aid of diagrams b) and c) in FIG. 3.

Here, in each case the sensor output Y_{G}′ at the limit value concentration XG is lower than the limit value output Y_{G} extrapolated in FIG. 1. In order however to be able to ensure a high degree of accuracy in the range of the limit value concentration X_{G} after the recalibration, an updated output line with a slope α′ has been drawn in diagram b) based on the limit value output Y_{G′} determined in the recalibration at the limit value concentration X_{G} and the initial zero point output Y₁ at the zero point X1, which has a smaller slope than the original output line shown in dashed lines in diagram b). Nevertheless, in the vicinity of the limit value concentration X_{G,} it can be determined with a high degree of accuracy based on a corresponding sensor output whether the limit value concentration X_{G} has been exceeded or undershot.

In a similar way, in diagram c) an updated output line was formed based on the limit value output Y_{G}′ determined during the recalibration at the limit value concentration X_{G} and the slope a of the output line determined in the initial calibration, which accordingly runs parallel to the original output line shown in dashed lines in diagram c). Also, by forming this updated output line, it can be ensured that in the vicinity of the limit value concentration X_{G} one can determine with a high degree of precision whether the value is exceeded or undershot, even if for example in this case the extrapolated zero point output Y₁′ deviates from the originally determined zero point output Y₁.

Accordingly, the limit value single-point calibration can take into account with less effort the reduction in sensor output over time in the recalibration according to diagrams b) and c), while at the same time it can determine with a high degree of accuracy whether a concentration of a gas to be detected in a gas mixture is above or below a predetermined limit value.

1. A method of calibrating at least one sensor unit provided for determining a concentration of a predetermined gas in a gas mixture, the method comprising:
performing an initial calibration of the at least one sensor unit at using at least two calibration gas mixtures, wherein a first calibration gas mixture has a first predetermined concentration of the predetermined gas, wherein a second calibration gas mixture has a second predetermined concentration of the predetermined gas, and wherein performing the initial calibration comprises:
determining an initial zero point output of the at least one sensor unit with the first calibration gas mixture in which the first predetermined concentration of the predetermined gas is zero;
determining a further output of the at least one sensor unit with the second calibration gas mixture in which the second predetermined concentration of the predetermined gas is greater than a limit value concentration; and
forming an output straight line of the at least one sensor unit based on the initial zero point output and the further output, wherein the output straight line has an initial line slope;
performing, after the initial calibration, a recalibration of the at least one sensor unit, wherein performing the recalibration comprises:
determining a limit value output of the at least one sensor unit with a limit value gas mixture having a concentration of the predetermined gas corresponding to the limit value concentration; and
forming an updated output straight line of the at least one sensor unit based on the limit value output and the initial zero point output.

2. The method according to claim 1, wherein the updated output straight line comprises a line slope and a zero point value, and wherein the method further comprises:
checking whether at least one of the line slope of the updated output straight line or the zero point value of the updated output straight line is within a predetermined value range.

3. The method according to claim 1, wherein the second predetermined concentration of the predetermined gas in the second calibration gas mixture corresponds to an upper limit of a measuring range of the at least one sensor unit.

4. The method according to claim 1, wherein performing the initial calibration further comprises:
determining a plurality of several further outputs using a plurality of additional calibration gas mixtures, each additional calibration gas mixture having different non-zero concentrations of the predetermined gas; and
forming a compensation output line of the at least one sensor unit based on the initial zero point output and the plurality of several further outputs.

5. The method according to claim 1, further comprising:
simultaneously calibrating a plurality of sensors, wherein each sensor of the plurality of sensors is provided to detect concentrations of a plurality of gases in a plurality of respective gas mixtures.

6. The method according to claim 1, wherein the gas mixture comprises breathing air and the predetermined gas comprises at least one of CO, CO₂ or O₂.

7. The method according to claim 1, further comprising:
performing a calibration check using a test gas mixture, wherein a concentration of the predetermined gas in the test gas mixture is above the limit value concentration.

8. The method according to claim 1, further comprising:
monitoring, using the at least one sensor unit, a compressor, and wherein during at least one of the initial calibration or the recalibration:
the compressor is switched off:
a flush valve is opened; or
a display for an operator is adjusted.

9. The method according to claim 1, further comprising:
monitoring a flow rate of the respective gas mixture by the at least one sensor unit.

10. The method according to claim 9, further comprising:
issuing, responsive to at least one of the flow rate falling below or exceeding a limit value for a predetermined period of time, a warning.

11. An analysis unit for gas mixtures, comprising:
at least one sensor unit designed to output sensor data representing a concentration of a predetermined gas in a gas mixture; and
a control unit operatively coupled to the at least one sensor unit and designed to determine, while in a working mode, the concentration of the predetermined gas based on the sensor data, wherein the control unit is switchable between the working mode and a calibration mode, and wherein while in the calibration mode, the control unit is configured to perform a calibration method comprising:
performing an initial calibration of the at least one sensor unit using at least two calibration gas mixtures, wherein a first calibration gas mixture has a first predetermined concentration of the predetermined gas, wherein a second calibration gas mixture has a second predetermined concentration of the predetermined gas-and wherein performing the initial calibration comprises:
determining an initial zero point output of the at least one sensor unit with the first calibration gas mixture in which the first predetermined concentration of the predetermined gas is zero;
determining a further output of the at least one sensor unit with the second calibration gas mixture in which the second predetermined concentration of the predetermined gas is greater than a limit value concentration; and
forming an output straight line of the at least one sensor unit based on the initial zero point output and the further output, wherein the output straight line has an initial line slope;
performing, after the initial calibration, a recalibration of the at least one sensor unit, wherein performing the recalibration comprises:
determining a limit value output of the at least one sensor unit with a limit value gas mixture having a concentration of the predetermined gas corresponding to the limit value concentration; and
forming an updated output straight line of the at least one sensor unit based on the limit value output and the initial zero point.

12. The analysis unit according to claim 11, wherein the control unit is further designed to issue, while in the working mode, at least one of a control command to open a flush valve or a control command to switch off a compressor responsive to detecting that a limit value concentration of the predetermined gas of the at least one sensor unit has been exceeded.

13. The analysis unit according to claim 11, wherein;
the analysis unit is provided with a gas reservoir containing a test gas mixture, wherein the test gas mixture includes the predetermined gas at a concentration above the limit value concentration; and
the control unit is designed to switch automatically to a test mode after at least one of a predetermined period of time or when the analysis unit is restarted, and wherein the control unit is designed to carry out a test of the at least one sensor unit using the test gas mixture contained in the gas reservoir.

14. The analysis unit according to claim 11, wherein the at least one sensor unit comprises an electrochemical sensor unit.

15. The analysis unit of claim 11, wherein the analysis unit is coupled to a compressor, wherein the compressor is configured for compressing breathing air for filling into a compressed gas reservoir, and wherein the analysis unit is designed to monitor an output of compressed breathing air for a concentration of at least one predetermined gas during operation of the compressor.

16. The analysis unit of claim 15, wherein the control unit of the analysis unitis operatively coupled to a control unit of the compressor.

17. The method of claim 1, wherein the limit value concentration defines a maximum concentration of the predetermined gas in the gas mixture.

18. The method of claim 1, wherein forming the updated output straight line of the at least one sensor unit is based on the limit value output and the initial straight line slope.

19. The method of claim 9, further comprising:
automatically stopping, responsive to at least one of the flow rate falling below or exceeding a limit value for a predetermined period of time, the calibration.

20. The analysis unit of claim 15, wherein the control unit of the analysis unit is integrally formed with a control unit of the compressor.
